(19) United States
(12) Patent Application Publication
(10) Pub. No.: US 2019/0247602 A1
(43) Pub. Date: Aug. 15, 2019

(54) CURVE STABILIZER FOR AN ENDOTRACHEAL TUBE

(71) Applicants: Drexel University, Philadelphia, PA (US); The Children's Hospital of Philadelphia, Philadelphia, PA (US)

(72) Inventors: Bryan John Melilli, Bensalem, PA (US); Vinay Moreshwar Nadkarni, Media, PA (US); Ian Yuan, Philadelphia, PA (US)

(21) Appl. No.: 16/275,934

(22) Filed: Feb. 14, 2019

Related U.S. Application Data
(60) Provisional application No. 62/630,874, filed on Feb. 15, 2018.

(51) Int. Cl. A61M 16/04

(52) U.S. Cl. CPC A61M 16/0497; A61M 16/0409; A61M 16/0434; A61M 16/0493

(57) ABSTRACT

An endotracheal tube is provided having flexible tubing of a predetermined inner diameter and a predetermined length adapted for endotracheal intubation via a mouth or nasal passage of a patient and a separate curve stabilizer to which the flexible tubing may be attached and through which the flexible tubing extends. The curve stabilizer includes walls forming a hollow elbow which has distal and proximal ends and a curved section therebetween and being sufficiently rigid to retain a portion of the flexible tubing in a curved shape forming an anti-kink bend at any selected location along the predetermined length of the flexible tubing. A curve stabilizing device and a method of endotracheal intubation are also disclosed.

### CROSS REFERENCE TO RELATED APPLICATIONS

This application claims the benefit under 35 USC § 119(e) of U.S. Provisional Patent Application No. 62/630,874, filed Feb. 15, 2018.

### BACKGROUND

Endotracheal tubes are inserted in patients to assist with ventilation, typically when the patient is under anesthesia or has a medical condition that requires artificial ventilation. Endotracheal tubes are available in many shapes, with the most common shapes being a flexible, straight tube (i.e., not formed with a fixed curve section) and a tube formed with a fixed relatively-sharp curved section (i.e., a curved tube).

By way of example, straight tubes may be used in an intensive care unit (ICU) or in an operating room (OR), for instance, for many surgical procedures involving a part of the body away from the head and neck. Generally, the straight tube has been used in the ICU because it is easier to insert a suction catheter into a straight tube than a curved tube, such as a so-called RAE (Ring, Adair, Elwyn) tube. Suction catheters are used to remove obstructions such as secretions from the patient. The curve of a RAE tube tends to require a smaller than ideal suction catheter in order to successfully navigate the curve of the tube.

Surgical procedures involving the head, neck, and mouth often require use of a fixed curved tube, which has a relatively-sharp preformed curve (i.e., is manufactured with a fixed curved portion) that allows the tube to bend around the patient's lower lip, thus diverting it away from the patient's face. This provides the surgeon with more room to operate and reduces the chance of the tube being inadvertently kinked or dislodged by the surgeon. An example of a curved tube is a so-called RAE (Ring, Adair, Elwyn) tube, which may be provided in a form for intubating through the mouth of the patient (i.e., an oral RAE) or in a form for intubating through the nose of the patient (i.e., a nasal RAE).

Conventionally, RAE tubes have a pre-formed curved section at a fixed location or position along the length of the tube that varies with each tube size (i.e., size of inner diameter of tube). This location is selected such that the tube will fit most patients. However, such tubes may not properly fit some patients due to variations in trachea length and diameter, which is typically related to the height and weight, respectively, of the patient.

By way of example, in a relatively tall and lean patient, a 5.0 mm internal diameter (ID) RAE tube would have an appropriate diameter based on age and weight, but may have a length that is too short. Selecting a larger size may result in an appropriate length of a tube, but the larger diameter may injure the vocal cords causing airway edema or may simply not fit through the vocal cords.

Conversely, in a relatively short and heavier patient, a 5.0 mm ID pre-formed curve tube may be an appropriate diameter, but the length of the tube may be too long, resulting in ventilating only one lung (mainstem intubation). In addition, for patients whose height and weight do not correlate, which is often the case in patients with congenital syndromes, endocrine abnormalities, growth deficiencies, and poor nutrition status, it may be difficult to provide an appropriate curved tube that provides a proper fit (diameter and length) to a particular patient. Thus, this can lead to a need for multiple intubations and tube changes to find the right combination of tube diameter versus tube length and can result in airway trauma, desaturations, and edema.

There are also occasions when a patient may have already been intubated with a straight tube but may need a RAE tube for a surgical procedure, or vice-versa, when a patient with a RAE tube may need to be transferred to the ICU after surgery, where a straight tube is preferred since it is easier to clean using suction catheters. There are well-known risks for complications associated with both intubation and extubation, so reducing or eliminating unnecessary tube exchanges should provide a great benefit to the patient.

By way of further example, a patient may be applied with a curved tube for a particular procedure and then may continue to require a tracheal tube after the procedure. Thus, the curved tube may need to be removed and replaced with a straight tube. As discussed above, there are significant issues arising from such a requirement for the use of different types of endotracheal tubes. For instance, multiple extubations and re-intubations while changing from one type of tube to another can put patients at risk for numerous complications, such as desaturation, apnea, airway obstruction, and airway edema. Patients with difficult airways may be at even greater risk from each airway manipulation.

As discussed above, curved tubes may be available in the same diameters as straight tubes, but each size of curved tube has its curvature located at a different fixed point along the longitudinal axis of the tube. Studies have found that these types of tubes may not always be a good fit for certain patients, including smaller patients. The location of the curve tends to fit average-sized healthy patients. However, as stated above, patients with congenital syndromes resulting in abnormal head and neck anatomy or in patients that have mismatching height and weight, the location of the preformed curve is typically not appropriate even though the inner diameter of the tube may be correct.

### BRIEF DESCRIPTION OF THE DRAWINGS

Various features of the embodiments described in the following detailed description can be more fully appreciated when considered with reference to the accompanying figures, wherein the same numbers refer to the same elements.

FIG. 1 is a diagram showing a conventional straight endotracheal tube positioned within a patient.

FIG. 2 is a perspective view of a conventional straight endotracheal tube.

FIG. 3 is a diagram showing a conventional curved tube (oral RAE) positioned within a patient.

FIG. 4 is a diagram showing a conventional curved tube (nasal RAE) positioned within a patient.

FIG. 5 is a perspective view of a conventional curved tube (oral RAE) (top of FIG. 5) and a perspective view of a conventional curved tube (nasal RAE) (bottom of FIG. 5).

FIG. 6 is a perspective view of a conventional curved tube (oral RAE) (left side of FIG. 6) adjacent a straight tube flexed to form a curve with a curve stabilizer device in accordance to an embodiment (right side of FIG. 6).

FIG. 7 is an elevational view of the curved portion of the straight tube with the curve stabilizer in accordance to an embodiment.

FIG. 8 is a left side elevational view of a curve stabilizer device defining a curve of an angle θ in accordance to an embodiment.

FIG. 9 is a diagram showing an endotracheal tube including the curve stabilizer device shown in FIG. 8 positioned relative to a patient.

FIG. 10 is a front elevational view of a curve stabilizer device of FIG. 8.

FIG. 11 is a left side elevational view of the curve stabilizer device of FIG. 8.

FIG. 12 is a right side elevational view of the curve stabilizer device of FIG. 8.

FIG. 13 is a perspective view of a left side of the curve stabilizer device of FIG. 8.

FIG. 14 is a perspective view of a right side of the curve stabilizer device of FIG. 8.

FIG. 15 is a magnified elevational view of a distal end of the curve stabilizer device of FIG. 8.

FIG. 16 is a magnified elevational view of a proximal end of the curve stabilizer device of FIG. 8.

### DETAILED DESCRIPTION

For simplicity and illustrative purposes, principles of embodiments are described herein by referring primarily to examples thereof. In the following description, numerous specific details are set forth to provide a thorough understanding of the embodiments. It will be apparent to one of ordinary skill in the art that the embodiments may be practiced without limitation to these specific details. In some instances, well known methods and structures have not been described in detail so as not to unnecessarily obscure the embodiments.

“Patient” or “subject” as used herein means a mammalian animal, including a human, a veterinary or farm animal, a domestic animal or pet, and animals normally used for clinical research. More specifically, the subject of these methods, systems and devices is a human.

According to an embodiment, a separately manufactured so-called curve stabilizer device is configured to attach to an otherwise relatively straight, but generally flexible, endotracheal tube for purposes of converting the tube into the shape of a desired fixed curved tube (such as an oral RAE tube or a nasal RAE tube) while ensuring the tube does not kink at the location of the curve. For this purpose, the curve stabilizer device supports both opposite lateral sides of the tube along the curved portion, thus blocking and preventing the sides of the tube from bulging outward and thereby preventing the formation of an undesired kink in the tube which might restrict airflow through the tube.

The curve stabilizer device, which is manufactured and formed separate of the flexible tubing, may be attached anywhere along the length of the tubing thereby permitting a curve to be custom positioned and formed in the tube at a location individually selected to precisely fit each patient regardless of their tracheal diameter and length. Additionally, use of the device permits a generally-straight flexible tube to be converted into a fixed curved tube (when the device is attached to the tube) and then returned to a flexible generally-straight tube (when the device is disconnected from the tube) thereby eliminating any need to change between fixed curved and generally-straight tubes for a patient already intubated before or after a procedure. Still further, the curve stabilizer device may serve as a bite block and/or an affixation device that can be used in the ICU separately from its applications in an operating room.

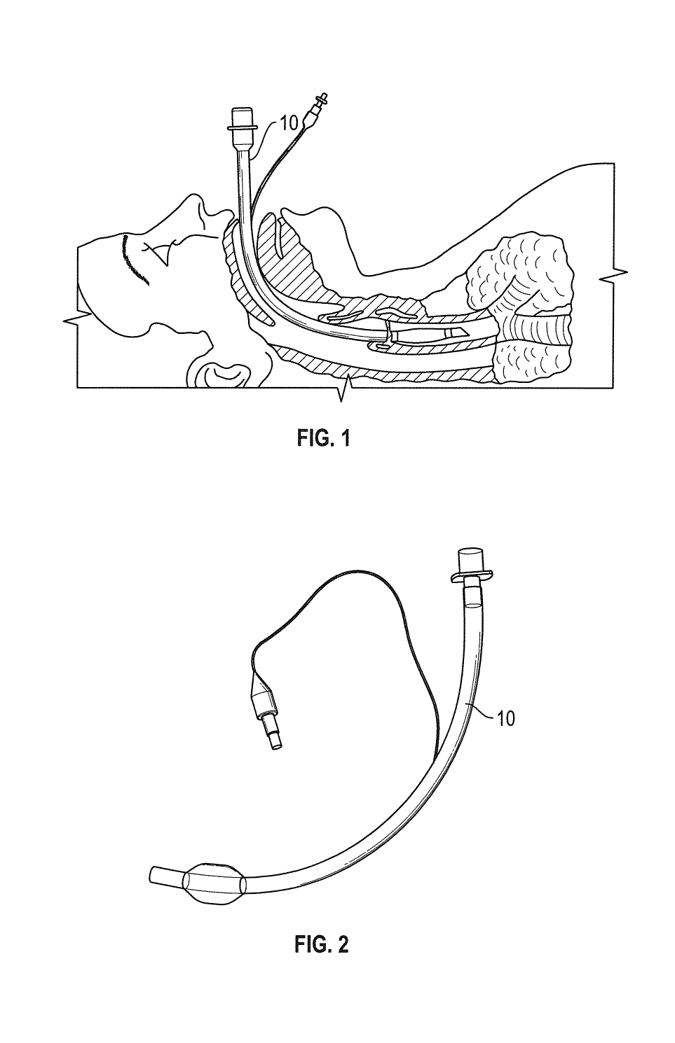

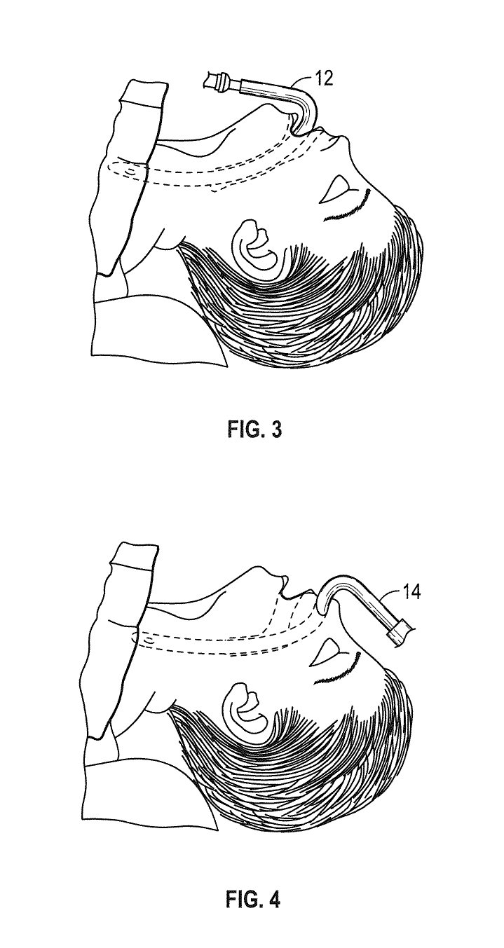

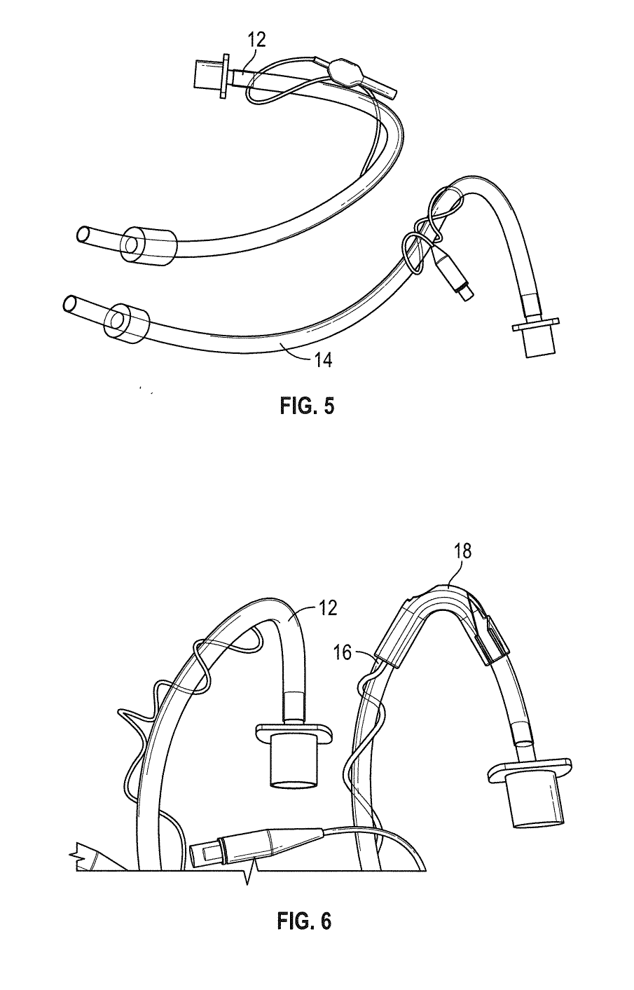

For purposes of comparison, an example of a conventional straight flexible tube 10 (i.e., a tube not manufactured to have a fixed curve section) is shown in FIGS. 1 and 2, an example of a fixed curved oral tube 12 (i.e., a tube manufactured to have a fixed curve section) is shown in FIG. 3 and the upper portion of FIG. 5, and an example of a fixed curved nasal tube 14 (i.e., a tube manufactured to have a fixed curve section) is shown in FIG. 4 and the lower portion of FIG. 5.

FIG. 6 provides an image of the fixed curved oral tube 12 discussed above adjacent a straight flexible tube 16 (similar to tube 10 shown in FIG. 2) but having a curve stabilizer device 18 according to an embodiment. The curve stabilizer device 18 is attached on and extends circumferentially about the tube 16 and results in the formation of a relatively sharp curve in the otherwise relatively-straight, flexible tube 16. More specifically, FIG. 6 shows the geometry of a 4.0 mm (inner diameter) oral RAE tube 12, and a curve stabilizer device 18 which fits cleanly onto an otherwise straight 4.0 mm (inner diameter) endotracheal tube 16. When the separate curve stabilizer device 18 is attached to the straight tube 16, the straight tube 16 takes on and is held in a shape that mimics the shape of a corresponding sized RAE tube, with the one possible exception being that straight tubes typically have a greater length than RAE tubes.

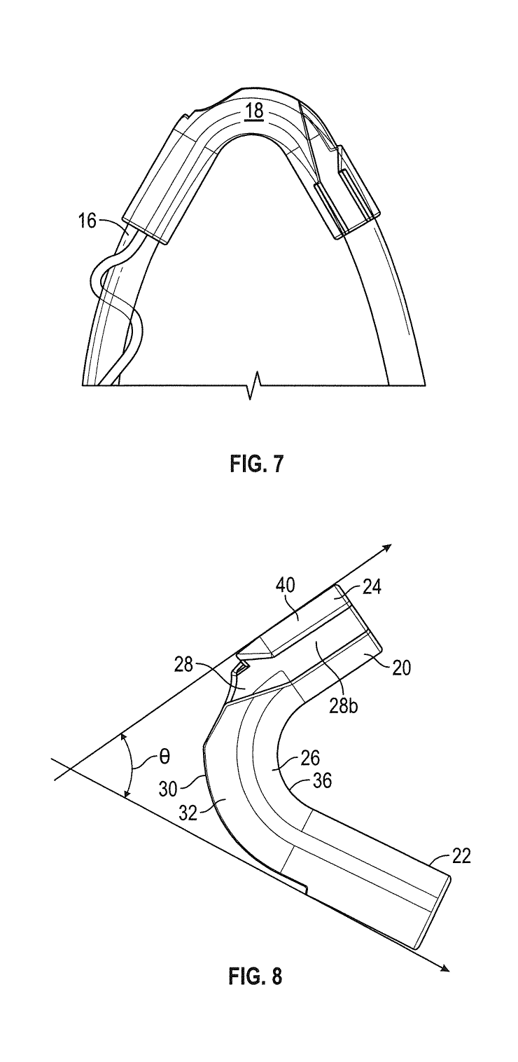

Accordingly, as shown in FIG. 6, the tube 16 is curved or bent into a shape that mimics that of the RAE tube 14 shown on the left side of FIG. 6. In addition to the device 18 allowing the straight tube 16 to mimic the fixed curve of the RAE tube 12, it also prevents kinking at the bend (i.e., along the curve). FIG. 7 provides a magnified image of the tube 16 and stabilizer 18 and shows that there is no kinking of the tube along the curve. Testing performed on a tube 16 and stabilizer 18 combination to measure airflow resistance through the tube 16 has demonstrated that airflow resistance is similar to that of a RAE tube 12 having a preformed fixed curve.

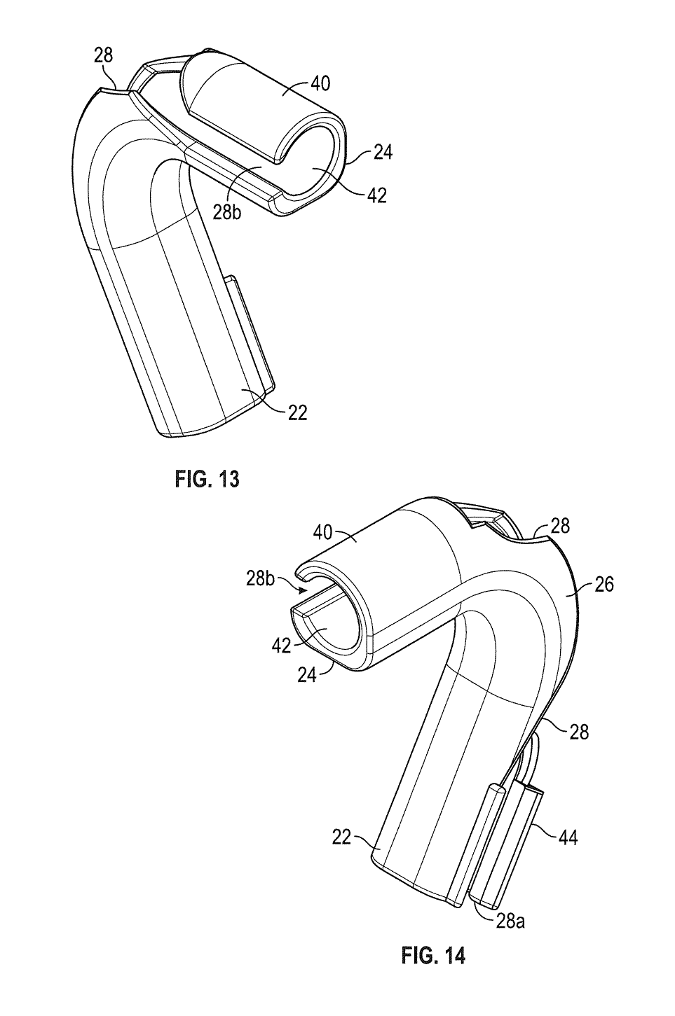

A contemplated embodiment of the configuration of a curve stabilizer device 20 is shown in FIGS. 8-16. The device 20 may be made of plastic or like material and may be formed using injection molding or like techniques. The illustrated embodiment of the curve stabilizing device 20 is in the form of a hollow elbow defining a cavity through which the flexible tubing extends. The walls of the elbow closely fit, abut and support the outer diameter of the tubing.

Preferably, the device 20 is sufficiently rigid to hold the angle at which a relatively straight flexible tube is to be bent. For instance, as best shown in FIG. 8, the angle “0” formed between the proximal and distal ends, 22 and 24 of the curve stabilizer device 20 is less than 90° (i.e., creating a bend of less than 90° in the otherwise straight tubing). For instance, the angle “0” shown in FIG. 8 may be between about 45° to about less than 900. The curved section 26 of the device 20 provides a smooth, anti-kink transition through the bend.

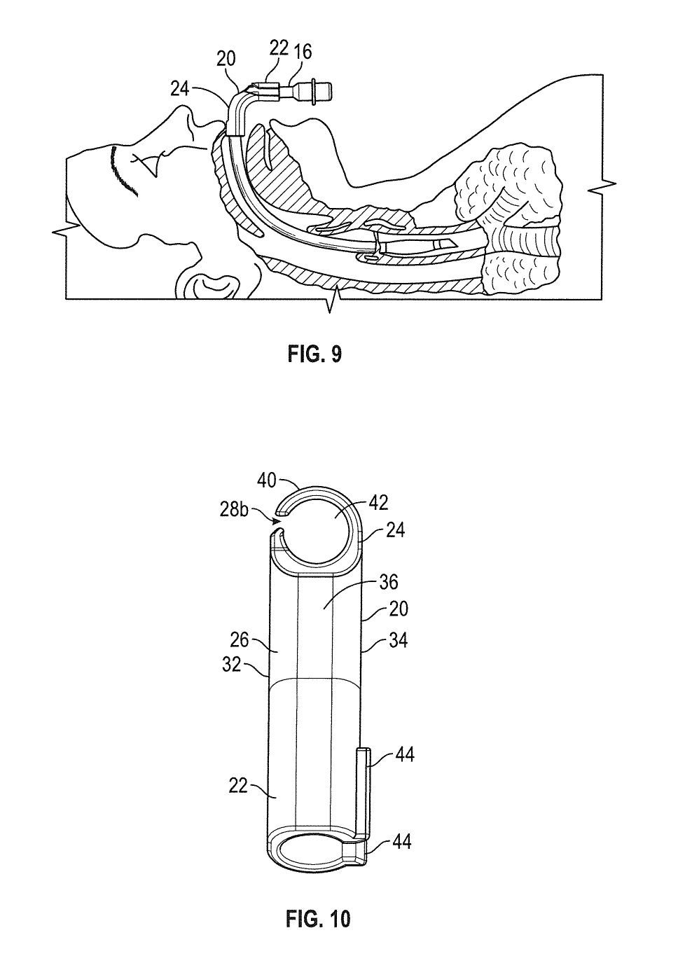

The proximal end 22 of the curve stabilizer device 20 is configured to extend externally of the patient's mouth and the distal end 24 is the portion of the curve stabilizer device 20 extending within the patient's mouth. For instance, see FIG. 9 showing the curve stabilizer device 20 installed on a length of flexible tubing 16 positioned within a patient in a manner similar to FIG. 1.

To enable the curve stabilizer device 20 to be snapped onto separately manufactured flexible tubing 16, the curve stabilizer device 20 has a tube insertion slot or channel 28 that extends continuously end-to-end throughout the length of the device 20 including through the curved section 26 and each of the proximal and distal ends, 22 and 24. The channel 28 permits the flexible tubing, such as tubing 16 discussed above, to be inserted and pushed into the hollow cavity defined by the walls of the curve stabilizer device 20 such that the tubing 16 extends within and through the curve stabilizer device 20 and is tightly gripped by the surrounding walls of the curve stabilizer device 20.

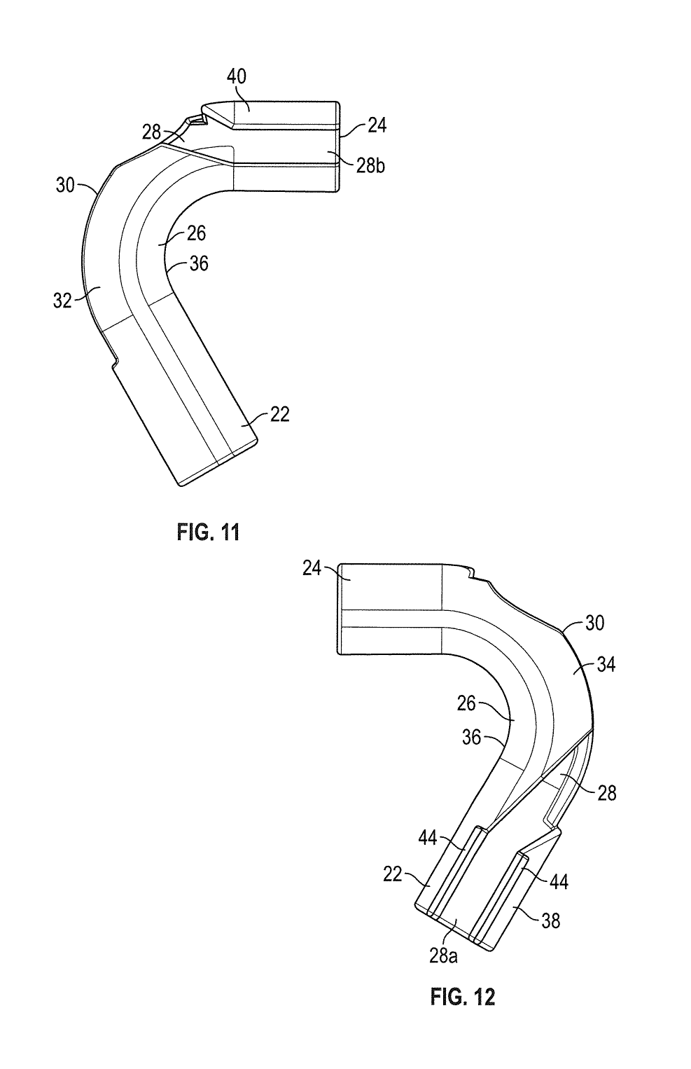

Preferably, the channel 28 extends uniformly on the outer radius 30 of the curved section 26. For instance, see FIGS. 11 and 12. Accordingly, the opposed sidewalls, 32 and 34, of the curved section 26 support both opposite sides of the flexible tubing throughout the curved portion 26, and thus blocks and prevents the sides of the flexible tubing tube from bulging outward. In addition, an inner radius wall 36 of the curved portion (see FIG. 10) supports and defines the curve of the flexible tubing through the curve stabilizer device 20. The walls 32, 34 and 36 prevent the formation of an undesired kink in the flexible tubing 16 within the curved section 26 which might otherwise restrict airflow through the tubing 16.

On the proximal and distal ends, 22 and 24, the channel 28 transitions to opposite sides of the curve stabilizer device 20. For instance, see channel sections 28a and 28b on opposite sides of the curve stabilizer device 20 shown in FIGS. 11 and 12. Accordingly, the channel 28 extending on the proximal and distal ends of the elbow are offset relative to the path of the channel extending through the outer radius 30 of the curved section 26. In addition, the path of the channel 28b extending through the distal end 24 extends on an opposite side of the elbow relative to the path of the channel 28a extending through the proximal end 22. Thus, the channel sections 28a and 28b define oppositely-extending locking tabs, 38 and 40 respectively, in the walls of the elbow-shaped curve stabilizer device 20. The locking tabs, 38 and 40, prevent release of the flexible tubing 16 after having been pressed through the channel 28, including channel sections 28a and 28b. In this regard, the width of the channel 28 and channel sections 28a and 28b need only be as large as is needed to press flexible tubing into and through the channel 28.

After the tubing has been pressed into the device 20, the walls of the curved section 26 and proximal and distal ends, 22 and 24, fix the flexible tubing at the curve and angle θ defined by the device 20 and support the walls of the tubing so as to provide an anti-kink function.

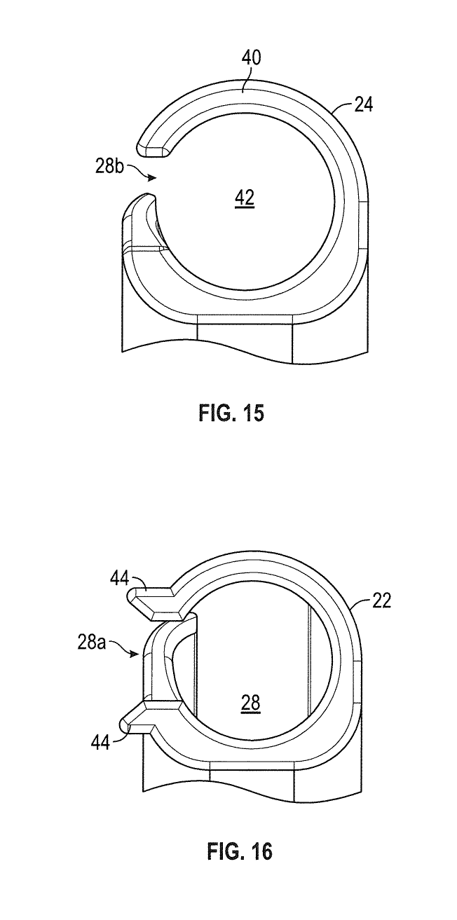

FIG. 15 provides a magnified view of the distal end 24 of the curve stabilizer device 20 (i.e., the end of the device 20 which is located in the mouth of the patient). As shown in FIG. 15, the walls forming the distal end 24 define the slot or channel 28b communicating with a cavity 42 that is substantially circular in transverse cross-section. The slot or channel 28b permits flexible tubing 16 to be pushed into the cavity 24 and enables the device 20 to be slid freely along the length of the flexible tubing to allow for ready positioning of the device 20 into an ideal and custom-fit location for the patient. The slot or channel 28b also permits the flexible tubing to be straightened for cleaning or like purposes without fully removing the device 20 from the flexible tubing. In this manner, the device 20 may be used as a fixation device for the flexible tubing to the patient. Thus, by removing the flexible tubing from the proximal end 22 of the device 20 only, the flexible tubing may be held in place by the distal end 24 which may also be located and function as a bite block for the flexible tubing.

FIG. 16 provides a magnified view of the configuration of the proximal end 22 of the device which is intended to be positioned externally of the patient's mouth. Unlike the smooth walls defining the slot or channel 28b, the slot or channel 28a in the proximal end 22 of the device 10 is defined by a pair of radially outward flared flanges 44 having tapered edges that provide a funnel-like configuration to enable easier insertion of flexible tubing through slot or channel 28a. The flanges 44 guide the flexible tubing into the proximal end 22 of the device 20 and aid in opening the slot or channel 28a by simple pushing of the flexible tubing in a direction into the device 20.

If desired, the distal end 24 of the device 20 may be extended, enlarged, or reinforced to form a more prominent bite block and the proximal end 22 may be provided with an additional flange or slot providing an affixation device so that the device 20, as well as the tubing connected to the device 20, may be firmly affixed to the face or chin of the patient.

Accordingly, embodiments disclosed herein provide a tube stabilizer device configured to readily convert the shape of a flexible, generally-straight tube to that of a fixed curved tube and thereby prevents any need for multiple tube changes and also positions the curve to custom fit each patient individually. This is accomplished while preventing the tube from developing a kink that may block airflow to the patient.

The tube stabilizer device is configured such that it can be quickly and easily attached to the outside (i.e., outer diameter wall) of an otherwise straight, flexible endotracheal tube, such as an oral or nasal tube, to create a curve of a similar shape as that of a preformed fixed curved tube of the same diameter, without allowing the tube to kink. The kinking is prevented by supporting the sides of the tube throughout the curve thereby preventing the sides of the tube from bulging outward and forming a kinked or collapsed shape. The presence of the curve stabilizer device on the tube prevents the tube from becoming deformed, thus keeping airflow unobstructed through the tube.

According to an embodiment, the tube stabilizer device, as discussed above, may be configured such that it may be manipulated with only a single hand of a caregiver while the other hand of the caregiver is free to hold the tube in a steady position. In this manner, accidental extubation while the device is being attached or detached from the tube may be prevented. Accordingly, the tube stabilizer device may be attached to a straight tube that has already been inserted in a patient. In this manner, the location of the curve can be properly positioned at an optimal location for the specific patient for ensuring that space is free for the surgeon during an operation or like procedure.

According to another contemplated embodiment, a distal end portion of the tube stabilizer device that is ultimately positioned inside the mouth of the patient may be attached to the tube prior to the tube being inserted into the patient. Thereafter, the tube stabilizer device may be slid along the length of the tube into a proper position before attaching the opposite proximal end of the curve stabilizer device to the tube which thereby causes the tube to assume a predetermined fixed curved shape.

According to these embodiments, tube changes should no longer be needed and the curve in the tube can be applied at an optimal location based on the length of the patient's trachea. This also provides a benefit of eliminating the need to stock multiple shapes of endotracheal tubes, as the curve stabilizer device would be capable of converting any straight tube into a fixed curved tube. Such a device could also be adapted to produce nasal curved tubes.

As discussed above, additional features of the curve stabilizer device may include the curve stabilizer device also serving as a bite block and/or affixation device to hold the tube in place on the patient for extended periods of time. These features would provide the device with additional utility of being useful in the ICU as well as the OR.

Bite blocks are used to protect the tube from a patient biting down on the tube, which could potentially cut off airflow or displace the tube. Accidental extubation can cause additional injury or other complications; thus, it is important to minimize the chances of such occurrence. The use of a pediatric bite block is typically avoided because they have not been found to be suitable in practice. However, a combination bite block and curve stabilizer device should overcome these problems.

Tube holders, also referred to as fixation devices, serve to keep a tube steady relative to the patient after it has been inserted in the patient. However, as is often the case for pediatrics, many of the devices are designed as smaller sizes of an adult device rather than being created specifically for children. This sometimes results in improper fitting. Embodiments of the curve stabilizer devices discussed herein may be located at the point where a tube enters the mouth and would likely extend the tube downward outside the lower lip and toward the chin in order to hold the tube into the curved shape, for instance, of a RAE tube. This curved portion of the device may be designed to serve as a fixation point onto the patient's face, thus allowing the curve to add another level of stability compared to conventional methods, which typically leave the free end of the tube extending forward.

As discussed above, fixed curved tubes are difficult to clean using suction catheters. In contrast, the embodiments discussed herein enable the separate curve stabilizing device to be able to be easily disconnected from the endotracheal tube to allow the tube to be straightened during cleaning. Afterward, the curve stabilizing device may be reconnected about the tube to reform the curve. In some embodiments, the curve stabilizer device may remain fixed to the patient's jaw with the end of the tube being capable of being slid within the curve stabilizer device or out of the curve stabilizer device without requiring the tube to be removed from the patient.

Accordingly, the curve stabilizing device should be able to bend a relatively straight length of relatively-flexible tubing into a shape that approximately matches a fixed curved oral or fixed nasal tube of the same diameter without allowing any kinks to form. Airflow through a straight tube bent with the curve stabilizer device should be comparable to airflow through a preformed fixed curved tube of the same size. The curve stabilizer device remains securely attached to the flexible tube even if the tube is moved or bumped during surgery.

Preferably, the curve stabilizer device is of a size that cannot be easily swallowed in the event that it is accidentally left in place once a surgical procedure has been completed (i.e., preferably, the device is not able to fit fully into a CPSC Small Object Choking Hazard testing tube). The curve stabilizer device may be provided in sizes that can accommodate the most commonly used tube inner diameters, for instance, in diameters ranging from about 3.0-7.0 mm in 0.5 mm increments. The device should be easy to use and attach and detach from a tube.

In addition to the above, the curve stabilizer device should not place excess pressure on the tube that would result in the tube becoming kinked, collapsed and/or obstructed. The amount of pressure that would cause obstruction may vary depending on tube diameter. Placement of the device may be limited to the outside of the tube in proximity of where a curve is desired along a length of tubing. Dimensions of the curve stabilizer device must be appropriate to support various tubes sizes and must not interfere with the function of the endotracheal tube, cuff, or pilot balloon.

Preferably, centimeter or like markings on the endotracheal tube are not completely covered and hidden by the curve stabilizer device. In addition, the curve stabilizer device may have a relatively low profile in at least certain locations to prevent it from being in the way of the surgeon, and the device must not be made of highly flammable materials due to its potential for use in close proximity with electrocautery equipment. Further, the material and shape of the curve stabilizer device must not result in unsafe pressures being placed on the patients face or lips. Few studies have been conducted on the pressures that would be damaging to the lips specifically, so it is presumed herein that it is not different from other parts of the body, and that therefore the pressure applied to the lower lip must be less than about 32 mmHg.

Attachment/detachment mechanisms of the device must be able to be operated with minimal effort so that one hand of a caregiver may hold the tube in place to prevent accidental extubation. A bite block portion of the curve stabilizer device should protect the tube while not damaging the patient's teeth.

While the principles of the invention have been described above in connection with specific devices, systems, and/or methods, it is to be clearly understood that this description is made only by way of example and not as limitation. One of ordinary skill in the art will appreciate that various modifications and changes can be made without departing from the scope of the claims below. Accordingly, the specification and figures are to be regarded in an illustrative rather than a restrictive sense, and all such modifications are intended to be included within the scope of the present invention.

We claim:

1. An endotracheal tube, comprising:
flexible tubing of a predetermined inner diameter and a predetermined length adapted for endotracheal intubation via a mouth or nasal passage of a patient; and
a separate curve stabilizer to which the flexible tubing may be attached and through which the flexible tubing extends;
the curve stabilizer including walls forming a hollow elbow which has distal and proximal ends and a curved section therebetween and being sufficiently rigid to retain a portion of the flexible tubing in a curved shape forming an anti-kink bend at any selected location along the predetermined length of the flexible tubing.

2. An endotracheal tube according to claim 1, wherein an angle (θ) formed between the distal and proximal ends of the elbow is between about 450 to less than about 900.

3. An endotracheal tube according to claim 2, wherein the walls forming the hollow elbow curve define a continuous open channel extending lengthwise along a full length of the curve stabilizer through the distal end, curved section, and proximal end of the curve stabilizer which permits the flexible tubing to be inserted into the curve stabilizer and removed from the curve stabilizer.

4. An endotracheal tube according to claim 3, wherein the channel includes an intermediate section that extends on an outer radius of the curved section and wherein walls of the curved section extend continuously along at least the opposite lateral side sections of the curved section such that the walls of the curved section abut, support, and prevent bulging and kinking of the adjacent walls of the tubing extending within the curved section of the curve stabilizer.

5. An endotracheal tube according to claim 4, wherein the channel includes opposite end sections extending through the proximal and distal ends of the curve stabilizer such that the opposite end sections of the channel are offset from the intermediate section of the channel and are offset and opposite from each other thereby forming locking tabs on both the proximal and distal ends of the curve stabilizer.

6. An endotracheal tube according to claim 5, wherein a portion of the channel extending on the proximal end of the curve stabilizer is defined by an opposed pair of radially outward extending flanges having tapered edges.

7. An endotracheal tube according to claim 1, wherein the curve stabilizer is injected molded and made of plastic.

8. An endotracheal tube according to claim 1, further comprising a bite block formed on the distal end of the curve stabilizer.

9. An endotracheal tube according to claim 1, further comprising an affixation device on the proximal end of the curve stabilizer.

10. An endotracheal tube according to claim 1, further comprising an inflatable cuff adjacent a distal end of the flexible tubing.

11. A curve stabilizer for an endotracheal tube, comprising walls forming a hollow elbow having distal and proximal ends and a curved section therebetween and being sufficiently rigid to retain a portion of flexible endtracheal tubing in a curved-shaped, anti-kinked bend at any selected location along the predetermined length of the flexible tubing, an angle formed between the distal and proximal ends of the elbow being between about 45° to about less than 900, and the walls of the hollow elbow including a continuous open channel extending lengthwise along a full length of the elbow through the distal end, curved section, and proximal end which permits the endotracheal tube to be inserted into the hollow elbow and removed from the hollow elbow.

12. A curve stabilizer according to claim 11, wherein the channel includes an intermediate section that extends on an outer radius of the curved section of the elbow and wherein walls of the curved section extend continuously along at least opposite lateral side sections of the curved section such that the walls of the curved section abut, support, and prevent bulging and kinking of adjacent walls of the endotracheal tube extending within the curved section.

13. A curve stabilizer tube according to claim 11, wherein the channel includes opposite end sections extending through the proximal and distal ends of the hollow elbow such that the opposite end sections of the channel are offset from the intermediate section of the channel and are offset and opposite from each other thereby forming locking tabs on both the proximal and distal ends of the hollow elbow.

14. A curve stabilizer according to claim 13, wherein a portion of the channel extending on the proximal end of the curve stabilizer is defined by an opposed pair of radially outward extending flanges having tapered edges.

15. A curve stabilizer according to claim 11, further comprising a bite block formed on the distal end of the hollow elbow and an affixation device on the proximal portion of the hollow elbow, and wherein the hollow elbow is injection molded of plastic.

16. An endotracheal intubation method, comprising the steps of:
attaching a separate elbow-shaped curve stabilizer to flexible endotracheal tubing at any location along a length of the flexible tubing to produce and retain a bend in the tubing and to define a length of the tubing inserted into a trachea of a patient; and
inserting the endotracheal tubing into the trachea of the patient.

17. An endotracheal intubation method according to claim 16, wherein the attaching step is performed after the inserting step.

18. An endotracheal intubation method according to claim 16, wherein the attaching step is performed before the inserting step.

19. An endotracheal intubation method according to claim 16, wherein a distal end of the elbow-shaped curve stabilizer is attached to the tubing before the inserting step and a proximal end of the elbow-shaped curve stabilizer is attached to the tubing after the inserting step.

20. An endotracheal intubation method according to claim 16, further comprising the steps of:
after the attaching and inserting steps, at least partially detaching the curve stabilizer from the tubing to permit the bend in the tubing to be straightened while the tubing remains inserted within the trachea of the patent; and
cleaning the tubing with a suction catheter after said detaching step.
